(19) United States
(12) Patent Application Publication
(10) Pub. No.: US 2024/0358765 A1
(43) Pub. Date: Oct. 31, 2024

(54) COMPOSITION FOR TREATING LIVER DYSFUNCTION

(71) Applicants: Terumo Kabushiki Kaisha, Tokyo (JP); Osaka University, Suita-shi Osaka (JP)

(72) Inventors: Hidetoshi EGUCHI, Suita-shi Osaka (JP); Yoshito TOMIMARU, Suita-shi Osaka (JP); Shigeru MIYAGAWA, Suita-shi Osaka (JP); Keisuke TOYA, Suita-shi Osaka (JP); Fumiya OHASHI, Ashigarakami-gun Kanagawa (JP)

(21) Appl. No.: 18/767,576

(22) Filed: Jul. 9, 2024

Related U.S. Application Data
(63) Continuation of application No. PCT/JP2023/000699, filed on Jan. 13, 2023.

(30) Foreign Application Priority Data
Jan. 14, 2022 (JP) 2022-004092

(51) Int. Cl. A61K 35/34; A61P 1/16; C12N 5/077

(52) U.S. Cl. CPC A61K 35/34; A61P 1/16; C12N 5/0658; C12N 2502/28

(57) ABSTRACT

A composition comprising skeletal myoblasts for treating liver dysfunction or improving liver function, in which the liver dysfunction can be hepatitis, liver fibrosis, liver cirrhosis, liver cancer, or liver failure. The composition can be used for promoting the proliferation of liver cells, regeneration of the liver tissue and/or angiogenesis, and providing a method for treating liver dysfunction, including a step of treating applying the composition to a site exhibiting liver dysfunction.

### CROSS-REFERENCE TO RELATED APPLICATIONS

The present disclosure is a continuation of and claims benefit to PCT/JP2023/000699 filed on Jan. 13, 2023, entitled “COMPOSITION FOR TREATING HEPATIC FUNCTION DISORDER” which claims priority to Japanese Patent Application No. 2022-004092 filed on Jan. 14, 2022. The entire disclosure of the applications listed above are hereby incorporated herein by reference, in their entireties, for all that they teach and for all purposes.

### BACKGROUND

The present disclosure relates to a composition for treating a liver dysfunction and others.

Recently, attempts have been made to implant various types of cells for repairing damaged tissues, etc. For example, for repairing myocardial tissue damaged by ischemic heart diseases such as angina pectoris and myocardial infarction, attempts have been made to use, e.g., fetal cardiomyocytes, skeletal myoblasts, mesenchymal stem cells, cardiac stem cells, ES cells and iPS cells. See, e.g., Concise Review: Cell Therapy and Tissue Engineering for Cardiovascular Disease, Haraguchi Y. et al., Stem Cells Transl. Med., 1 (2), 136-141 (2012).

As part of these attempts, a cell construct formed on a scaffold and a sheet-shaped cell culture, which is a sheet formed of cells, have been developed and described in Japanese Patent Publication No. 2007-528755 A and in the paper entitled “Tissue Engineered Myoblast Sheets Improved Cardiac Function Sufficiently to Discontinue LVAS in a Patient with DCM: Report of a Case” by Yoshikawa Sawa, et al. (i.e., Tissue Engineered Myoblast Sheets Improved Cardiac Function Sufficiently to Discontinue LVAS in a Patient with DCM: Report of a Case, Sawa Y. et al., Surg. Today, 42 (2), 181-184 (2012)).

For the application of the sheet-shaped cell culture to treatment, for example, studies have been made on use of an epidermal sheet-shaped cell culture for treating skin damages caused by, e.g., burn; use of a corneal epithelial sheet-shaped cell culture for corneal damage; and use of an oral mucosa sheet-shaped cell culture for esophageal cancer endoscopic resection. Some of them have entered the stage of clinical application.

As one of the applications, it has been proposed to use a sheet-shaped cell culture for treating damage to organs such as digestive tract. For example, International Patent Publication No. WO/2017/130802 discloses the use of a sheet-shaped cell culture containing mesenchymal stem cells for treating or preventing leakage from a damaged portion of the gastrointestinal tract caused by suture failure, etc.

Furthermore, International Patent Publication No. WO/2021/149830 discloses an engraftment sheet material having satisfactory engraftment efficiency to the surface of an organ and satisfactory operability in clinical applications. More specifically, it has been demonstrated that a myoblast sheet, which has an extracellular matrix layer on one of the surfaces and a biodegradable gel layer on the other surface, can be satisfactorily engrafted in the surface of the liver or colon by implanting the sheet such that the surface the extracellular matrix layer faces the organ.

### BRIEF SUMMARY

It is an object of the present disclosure to provide a solution for treating liver dysfunction.

The present inventors have studied a method for treating liver dysfunction. During the studies, the inventors of the present disclosure focused on the possibility of treating liver dysfunction by cytokines produced from cells. Then, the inventors conducted studies with a view to demonstrating the possibility. As a result, the inventors found that the proliferation of the liver cells, regeneration of the liver tissue and angiogenesis are promoted by cytokine produced from skeletal myoblasts. Based on their findings, the inventors conducted further studies, with the result that the present disclosure is accomplished.

More specifically, the present disclosure at least relates to the following:
[1] A composition comprising skeletal myoblasts for treating liver dysfunction or improving liver function.
[2] The composition according to [1], in which the liver dysfunction is hepatitis, liver fibrosis, liver cirrhosis, liver cancer, or liver failure.
[3] The composition according to [1] or [2], in which the liver dysfunction is liver cirrhosis.
[4] The composition according to [1] or [2], in which the liver dysfunction is liver cancer.
[5] The composition according to [4], for application to the liver after liver cancer resection.
[6] The composition according to any one of [1] to [5], for promoting proliferation of liver cells, regeneration of the liver tissue and/or angiogenesis.
[7] The composition according to any one of [1] to [6], for treating liver dysfunction through cytokine production.
[8] A method for treating liver dysfunction, including a step of applying the composition according to any one of [1] to [7] to a site exhibiting liver dysfunction.
[9] The method according to [8], in which the step of applying the composition to a site exhibiting liver dysfunction is performed by an implantation device.
[10] The method according to [9], in which the implantation device is a catheter.

According to at least one embodiment of the present disclosure, it is possible to treat liver dysfunction such as hepatitis, liver fibrosis, liver cirrhosis, liver cancer, or liver failure. In particular, according to the present disclosure, it is possible to treat terminal-stage decompensated cirrhosis whose improvement cannot be expected by current internal therapy as well as to treat liver failure of residual liver after liver cancer resection. Furthermore, according to the present disclosure, it is possible to promote proliferation of the liver cells, regeneration of the liver tissue and/or angiogenesis. Moreover, according to the present disclosure, it is possible to treat liver dysfunction through cytokine production.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 shows charts of the production of growth factors derived from human skeletal muscle myoblasts, where each individual value is an average value (n=3) and an error bar represents a standard deviation in accordance with embodiments of the present disclosure.

FIG. 2 shows the procedure of inducing the capillary formation of immortalized human liver endothelial cells (TMNK-1) by a factor derived from human skeletal muscle myoblasts (HSMM) in accordance with embodiments of the present disclosure.

FIG. 3 shows optical microscope images (1 and 5 hours after the start of culture) of capillary formation of TMNK-1 induced by a factor derived from human skeletal muscle myoblasts, where, in the regions surrounded by a circle, capillary formation of TMNK-1 is induced by a factor derived from human skeletal muscle myoblasts in accordance with embodiments of the present disclosure.

FIG. 4A shows optical microscope images (24 hours after the start of culture) of capillary formation of TMNK-1 induced by a factor derived from human skeletal muscle myoblasts in accordance with embodiments of the present disclosure.

FIG. 4B shows a chart of the total length of a capillary observed in the capillary formation of TMNK-1 induced by a factor derived from human skeletal muscle myoblasts of FIG. 4A, where each individual value is an average value of treated group 1 (n=3), treated group 2 (n=3), and control group (n=3), respectively, and an error bar represents a standard deviation.

FIG. 4C shows a chart of the number of branches of the capillary in the capillary formation of TMNK-1 induced by a factor derived from human skeletal muscle myoblasts of FIG. 4A, where each individual value is an average value of treated group 1 (n=3), treated group 2 (n=3), and control group (n=3), respectively, and an error bar represents a standard deviation.

### DETAILED DESCRIPTION

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Unless otherwise specified, all technical terms and scientific terms used herein have the same meaning that those skilled in the art commonly understand. All patents, applications and other publications and information cited herein are incorporated herein by reference in their entirety.

It is with respect to the above issues and other problems that the embodiments presented herein were contemplated.

### Composition for Treating Liver Dysfunction:

At least one aspect of the present disclosure relates to a composition comprising skeletal myoblasts for treating liver dysfunction or improving liver function (sometimes referred to as “the composition of the present disclosure”). In an embodiment, liver dysfunction is hepatitis, liver fibrosis, liver cirrhosis, liver cancer, or liver failure. In an embodiment, liver dysfunction is liver cirrhosis. In an embodiment, liver dysfunction is liver cancer. In an embodiment, the composition of the present disclosure is used for application to the liver after liver cancer resection. In an embodiment, the composition of the present disclosure is used for promoting the proliferation of the liver cells, regeneration of the liver tissue and/or angiogenesis. In an embodiment, the composition of the present disclosure is used for treating liver dysfunction through cytokine production.

The liver is an organ having a wide variety of functions such as metabolism, maintenance of body-fluid homeostasis, and digestion as well as regeneration ability. In the liver, blood flows in from the hepatic artery and portal vein, passes through the central vein and flows out from the hepatic vein. The liver tissue is constituted of a mass of liver lobules and the central vein passes through the central shaft part of a liver lobule. The liver cells are radially arranged around the central vein to form the liver cell plate and occupy most of the whole cells in the liver. In the specification, the “liver cells” are interchangeably used with “hepatic parenchymal cells”. The cells other than the liver cells in the liver are called “liver non-parenchymal cells”. Examples thereof include sinusoidal endothelial cells, Kupffer cells, dendritic cells, natural killer (NK) cells, hepatic stellate cells, and monocyte-derived macrophages.

In the present disclosure, the liver is not limited as long as it is the liver of a living organism. Examples of the liver may include, but are in no way limited to, the liver of a human, a non-human primate, a rodent (such as a mouse, a rat, hamster or a guinea pig) and a mammal such as a rabbit, a dog, a cat, a pig, a horse, a cow, a goat or sheep.

In the present disclosure, the term “liver function” refers to a function of a healthy liver such as metabolism, maintenance of body-fluid homeostasis and digestion. In the present disclosure, the term “liver dysfunction” refers to inhibition, suppression and/or impairment of the liver function due to some cause. For example, the liver dysfunction refers to hepatitis, liver fibrosis, liver cirrhosis, liver cancer, or liver failure. In the present disclosure, the term “healthy liver” refers to an unharmed (intact) liver not influenced by, e.g., a disease, a disorder and/or a treatment.

In the present disclosure, the term “skeletal myoblasts” refers to myoblasts present in the skeletal muscle. The skeletal myoblasts are commonly known in the technical field and can be prepared from the skeletal muscle by any one of the methods commonly known (for example, a method disclosed in Japanese Patent Publication No. 2007-89442) or is commercially available (for example, Human Skeletal Muscle Myoblasts (HSMM) sold by Lonza, Cat #CC-2580). Skeletal myoblasts may be identified by a maker. Examples of the marker include, but are not limited to, CD56, α7 integrin, myosin heavy chain IIa, myosin heavy chain IIb, myosin heavy chain IId (IIx), MyoD, Myf5, Myf6, myogenin, desmin and PAX3. In a specific embodiment, skeletal myoblasts are CD56 positive. Further, in a specific embodiment, skeletal myoblasts are CD56 positive and desmin positive. Skeletal myoblasts may be derived from any organism having skeletal muscle. Examples of the organism include, but are not limited to, a human, a non-human primate, a rodent (such as a mouse, a rat, a hamster or a guinea pig) and a mammal such as a rabbit, a dog, a cat, a pig, a horse, a cow, a goat or sheep. In an embodiment, the skeletal myoblasts refer to mammalian skeletal myoblasts. In a specific embodiment, the skeletal myoblasts refer to human skeletal myoblasts. Furthermore, the skeletal myoblasts can be collected from any skeletal muscle. In an embodiment, skeletal myoblasts refer to skeletal myoblasts derived from the thigh, neck or abdomen.

In the present disclosure, the “composition comprising skeletal myoblasts” refers to a composition comprising at least skeletal myoblasts and an additional component having biocompatibility. Herein, the additional component having biocompatibility refers to a component having no undesirable effect, such as an inflammatory response, an immune response or a toxic reaction, on living body tissues and cells, or having such an effect but at least the effect is low. Examples of the additional component may include, but are not particularly limited to, a pharmaceutical acceptable carrier, an excipient, cells other than skeletal myoblasts, a component enhancing, e.g., viability, engraftment and/or functionality of the composition of the present disclosure, a component useful for reproduction and/or healing of living body tissues or promotion thereof, and/or a graft. It should be appreciated that those skilled in the art, who are familiar with the additional components and after reading the present disclosure, could appropriately select an additional component commonly known and use it. Furthermore, the aforementioned additional components can be used in combination with the composition of the present disclosure.

The composition comprising skeletal myoblasts described herein, although the state thereof is not particularly limited, exhibits, for example, a liquid-state having flowability, such as an aqueous solution, a suspension or an emulsion or a solid state having flexibility such as a sheet-shaped cell culture. Particularly, in view of application to the liver surface, the composition comprising skeletal myoblasts may be arranged as a sheet-shaped cell culture. In an embodiment, the composition comprising skeletal myoblasts is a pharmaceutical composition.

In the present disclosure, the “sheet-shaped cell culture” refers to a sheet formed of cells connected to each other. Cells may be mutually connected directly (e.g., including cells connected via a cellular element such as an adhesion molecule) and/or via an intervening substance. The intervening substance is not particularly limited as long as it can connect cells at least physically (e.g., mechanically). Examples thereof include an extracellular matrix. The intervening substance may correspond to a cell-derived substance and particularly a substance derived from cells constituting a cell culture. Cells are at least physically (e.g., mechanically) connected and further may be functionally, for example, chemically or electrically connected. The sheet-shaped cell culture may be composed of a single cell layer (e.g., uni-layer), two or more cell layers (e.g., a laminate (multilayer), for example, 2 layers, 3 layers, 4 layers, 5 layers or 6 layers). Furthermore, the sheet-shaped cell culture may not have a clear layer-structure of cells but may have a three-dimensional structure having a thickness beyond the thickness of a single cell. For example, the cells may not necessarily be uniformly arranged in the horizontal direction, that is, the cells may be non-uniformly arranged in the vertical section of a sheet-shaped cell culture.

In at least one embodiment, the sheet-shaped cell culture of the present disclosure may be extremely fragile. For instance, it may be difficult to handle the sheet-shaped cell culture of the present disclosure. Accordingly, the sheet-shaped cell culture of the present disclosure may further have a reinforcement layer for the purpose of making handling easier and reducing a risk of breakage (e.g., when compared to a sheet-shaped cell culture without a reinforcement layer). The reinforcement layer is not limited as long as it does not impair the function of the sheet-shaped cell culture of the present disclosure and reinforces the structure thereof. The reinforcement layer may comprise, for example, a gel and/or a polymer. However, since the sheet-shaped cell culture of the present disclosure is to be implanted into a living body, the reinforcement layer may correspond to a biocompatible reinforcement layer comprising, e.g., a biocompatible gel or polymer.

The gel to be used in the reinforcement layer, for example arranged as a biocompatible gel, is not limited as long as it does not adversely affect a living body when introduced. Examples of the gel may include, but are not limited to, fibrin gel, fibrinogen gel, gelatin gel and collagen gel.

The polymer to be used in the reinforcement layer, for example arranged as a biocompatible polymer, is not limited as long as it does not adversely affect a living body when introduced. Examples of the polymer may include, but are not limited to, polylactic acid, polydioxano, polyglycapro and collagen.

In a case where a sheet-shaped cell culture having a reinforcement layer is applied to an application site, the sheet may be applied such that the reinforcement layer is not directly arranged in contact with the site. In other words, the sheet-shaped cell culture may be applied such that the sheet-shaped cell culture is positioned between an application site and the reinforcement layer.

The composition of the present disclosure is applied to the liver to treat liver dysfunction or improve liver function. In at least one embodiment, the composition of the present disclosure is applied to the liver surface to treat liver dysfunction or improve liver function. In an embodiment, the composition of the present disclosure is applied to the liver by an implantation device to treat liver dysfunction or improve liver function. In an embodiment, the composition of the present disclosure is applied to the surface of the liver by an implantation device to treat liver dysfunction or improve liver function.

In the present disclosure, the “surface of the liver” refers to any surface of a healthy liver or liver influenced in some way by, e.g., a disease, a disorder, and/or a treatment. In an embodiment, the liver surface is the surface of the residual liver after partial hepatectomy (for example, liver cancer resection). In an embodiment, the liver surface is the surface (having a fibrosis region) of the liver exhibiting liver fibrosis.

In the present disclosure, the “application” refers to using the composition of the present disclosure to a target site, for example, adding, administrating, injecting, delivering, applying, attaching, engrafting, and/or implanting the composition of the present disclosure to a target site.

In the present disclosure, the “implantation device” refers to a medical device enabling the composition of the present disclosure to apply to the liver. In an embodiment, the implantation device refers to a medical device enabling the composition of the present disclosure to apply to the liver surface. In a case where the composition of the present disclosure is arranged as a sheet-shaped cell culture, the implantation device is, for example, a medical device having a planar structure capable of supporting and detaching the sheet-shaped cell culture. In view of operating it within the abdominal cavity, the implantation device may have a form, or structure, that can be inserted and passed through a cylindrical body which has been inserted in a body cavity in endoscopic surgery.

In the present disclosure, the term “hepatitis” refers to an inflammation in the liver. Examples of hepatitis include viral hepatitis (such as hepatitis A, B and C), alcoholic hepatitis, nonalcoholic steatohepatitis, drug-induced hepatitis and autoimmune hepatitis. Hepatitis is roughly divided into acute hepatitis and chronic hepatitis. Acute hepatitis is hepatitis that temporally occurs, whereas chronic hepatitis is hepatitis lasting for a long time, with the result that, e.g., a fibrotic change of the liver, and degeneration and necrosis of liver cells, may occur. Chronic hepatitis develops, through, e.g., a fibrotic change of the liver and degeneration and necrosis of liver cells, into liver fibrosis and liver cirrhosis, and then, the liver cancer.

In the present disclosure, the term “liver fibrosis” refers to the formation of a scar tissue in the liver, which is developed by replacing the parenchymal tissue of a healthy liver for connective tissue by a fibrotic change of the liver, and degeneration and necrosis of liver cells caused by repetitive damage and chronic hepatitis, followed by accumulating an extracellular matrix component such as collagen.

In the present disclosure, the term “liver cirrhosis” refers to the formation of a large amount of scar tissue in the liver, which is developed by replacing the parenchymal tissue of a healthy liver for a large amount of connective tissue by a fibrotic change of the liver, and degeneration and necrosis of liver cells caused by repetitive damage and chronic hepatitis followed by accumulating a large amount of an extracellular matrix component such as collagen. Liver cirrhosis in the initial stage having no symptoms is called compensated cirrhosis (a failure caused in part of the liver can be compensated by the remaining part). In contrast, liver cirrhosis in the terminal stage having a symptom such as jaundice, spider hemangioma and palmar erythema, which appears as a disease state progresses, is called decompensated cirrhosis. Compensated cirrhosis and initial-stage decompensated cirrhosis can be treated by current internal therapy (e.g., interferon, glycyrrhizin preparation, diuretic, albumin preparation and diet remedy). In contrast, in the terminal-stage decompensated cirrhosis, no improvement thereof is expected by current internal therapy. After the disease progresses into liver cancer and liver failure, there is no choice but a surgical treatment.

In at least one embodiment, the composition of the present disclosure is used for treating compensated cirrhosis or decompensated cirrhosis. In an embodiment, the composition of the present disclosure is used for treating initial- or terminal-stage compensated cirrhosis or decompensated cirrhosis.

In the present disclosure, the term “liver cancer” refers to a malignant tumor arising in the liver. Liver cancer is roughly divided into primary liver cancer and metastatic liver cancer. The primary liver cancer is mostly hepatocellular carcinoma (malignant tumor derived from liver cells). Hepatocellular carcinoma often results from progression to liver cancer through chronic hepatitis, liver fibrosis, liver cirrhosis.

In the present disclosure, the term “liver failure” refers to a significant reduction in the function of healthy liver, such as metabolism, body-fluid homeostasis and digestion, due to damage to most of the liver. Examples of the cause for liver failure include viral hepatitis, liver cirrhosis, alcoholic liver injury and drug-induced liver injury. Furthermore, the residual liver after partial hepatectomy (e.g., after liver cancer resection) sometimes shows liver failure. Liver failure is roughly classified into acute liver failure and chronic liver failure. Acute liver failure quickly progresses in a short period of time, whereas chronic liver failure gradually progresses over a long period of time.

In the present disclosure, the phrase “applied to the liver after liver cancer resection” refers to applying the composition of the present disclosure to the residual liver after liver cancer is partially or completely removed. In an embodiment, the composition of the present disclosure is applied to the surface of the residual liver after liver cancer is partially or completely removed. In an embodiment, the composition of the present disclosure is applied by an implantation device to the surface of the residual liver after liver cancer is partially or completely removed. In an embodiment, the liver after liver cancer resection shows liver failure.

In the present disclosure, the term “liver cells” refer to cells radially arranged around the central vein of a liver lobule in the liver to form the liver cell plate and made up of a majority of the whole cells in the liver. In the specification, the “liver cells” are interchangeably used with “hepatic parenchymal cells”.

In the present disclosure, the term “liver tissue” refers to a tissue constituted of a mass of liver lobules.

In the present disclosure, the term “angiogenesis” refers to a phenomenon where new blood vessels are formed from pre-existing vessels in, e.g., the liver and branched to construct a vascular network.

In the present disclosure, the term “cytokine production” refers to producing cytokines such as vascular endothelial growth factor (VEGF) and hepatocyte growth factor (HGF), which play a role in, e.g., angiogenesis, protection and repairment of cells. For example, in the liver to which the composition of the present disclosure is applied, cytokines such as VEGF and HGF, which play a role in, e.g., angiogenesis, protection and repairment of cells, are produced.

The composition of the present disclosure may comprise optional cells in addition to skeletal myoblasts. The optional cells are not particularly limited and may include, for example, adhesion cells (adherent cells). For example, adhesion cells (adherent cells) are included. Examples of the adhesion cells may include, but are in no way limited to, adhesive somatic cells (for example, cardiomyocytes, fibroblasts, epithelial cells, endothelial cells, liver cells, pancreatic cells, kidney cells, adrenal gland cells, periodontal ligament cells, gingival cells, periosteal cells, skin cells, synovial cells, cartilage cells) and stem cells (for example, tissue stem cells such as myoblasts, cardiac stem cells, pluripotent stem cells such as embryonic stem cells, iPS (induced pluripotent stem) cells and mesenchymal stem cells). The somatic cells may be differentiated from stem cells, particularly iPS cells (iPS cell-derived adherent cells). Non-limiting examples of cells include myoblasts, mesenchymal stem cells (for example, cells derived from bone marrow, fat tissue, peripheral blood, skin, hair root, muscle tissue, endometrium, placenta, umbilical cord blood), cardiomyocytes, fibroblasts, cardiac stem cells, embryonic stem cells, iPS cells, synovial cells, cartilage cells, epithelial cells (for example, oral mucosal epithelial cells, retinal pigment epithelial cells, nasal mucosal epithelial cells), endothelial cells (for example, vascular endothelial cells), liver cells (for example, hepatic parenchymal cells), pancreatic cells (for example, pancreatic islet cells), kidney cells, adrenal gland cells, periodontal ligament cells, gingival cells, periosteal cells, and skin cells. Non-limiting examples of the iPS cell-derived adherent cells include iPS cell-derived cardiomyocytes, fibroblasts, epithelial cells, endothelial cells, liver cells, pancreatic cells, kidney cells, adrenal gland cells, periodontal ligament cells, gingival cells, periosteal cells, skin cells, synovial cells, and cartilage cells.

The cells in the composition of the present disclosure may be derived from an organism that can be treated with the composition of the present disclosure. Examples of the organism include, but are not limited to, a human, a non-human primate, a dog, a cat, a pig, a horse, a goat, sheep, a rodent (for example, a mouse, a rat, a hamster, a guinea pig) and a rabbit. Furthermore, the number of types of cells in the composition of the present disclosure is not particularly limited. The composition may be constituted of only one type of cell or two types or more of cells. In a case where the number of types of cells in the composition is two or more, the content ratio (e.g., purity) of the most common type of cell is 50% or more, preferably 60% or more, more preferably 70% or more, and further preferably 75% or more, at the time when completion of the formation of the composition of the present disclosure. In embodiments, the most common type of cell is myoblasts, or more particularly skeletal myoblasts.

In an embodiment, the cells are autogenic cells. In a case where the composition of the present disclosure is used for implantation, the “autogenic cells” herein refer to cells derived from a recipient.

In an embodiment, the cells are isogenic cells. Herein, the “isogenic cells” refer to cells derived from the same species as the recipient and similar species based on the genetical relatedness (in other words, no rejection reaction occurs after implantation to the recipient). For example, in a case where the recipient is a human, human cells of the similar species based on the genetical relatedness correspond to isogenic cells.

In an embodiment, the cells are allogeneic cells. Herein, the “allogeneic cells” refer to cells derived from an organism belonging to the same species as the recipient but different species based on the genetical relatedness (in other words, a rejection reaction occurs after implantation to the recipient). For example, in a case where the recipient is a human, human cells of the different species based on the genetical relatedness correspond to allogeneic cells.

In the specification, the “autogenic cells” are used interchangeably with “autogenic cells”, and “isogenic cells” and “allogeneic cells” are interchangeably used with “xenogenic cells”.

In an embodiment, cells are heterogenic cells. In a case where the sheet-shaped cell culture is used for implantation, the “heterogenic cells” herein refer to cells derived from an organism of different species from the recipient. For example, in a case where the recipient is a human, cells derived from a monkey or a pig correspond to the heterogenic cells.

The autogenic cells and isogenic cells are preferable in the present disclosure because no rejection reaction occurs even if they are implanted. However, it is possible to use allogeneic cells and heterogenic cells. In a case where the allogeneic cells and heterogenic cells are used, an immunosuppressive treatment is often required to suppress a rejection reaction.

In a case where skeletal myoblasts are prepared from the striated muscle tissue, fibroblasts are contained in the cell population thus prepared. In a case where the composition of the present disclosure is produced using a cell population comprising skeletal myoblasts prepared from the striated muscle tissue, the cell population comprises a predetermined amount of fibroblasts. Fibroblasts are well known in the technical field and can be identified by a marker such as TE-7 (see, for example, Rosendaal et al., J Cell Sci. 1994; 107 (Pt 1): 29-37, Goodpaster et al., J Histochem Cytochem. 2008; 56 (4): 347-58).

In at least one embodiment, cells in the composition of the present disclosure include skeletal myoblasts prepared from striated muscle tissue. Accordingly, the cell population to be used in producing the composition of the present disclosure may comprise skeletal myoblasts and fibroblasts. In an embodiment, the cell population to be used in producing the composition of the present disclosure has a CD56 positive rate of 50% or more, 60% or more, 65% or more, 70% or more, 75% or more, 80% or more, 85% or more or 90% or more, and preferably 60% or more.

The cell population to be used in producing the composition of the present disclosure may comprise fibroblasts. However, in a case where the content rate of fibroblasts is too high is not preferable since the content rate of the skeletal myoblasts decreases. Therefore, in one embodiment, the cell population used in the production of the composition of the present disclosure may have a TE7 positive rate of 50% or less, 40% or less, 35% or less, 30% or less, 25% or less, 20% or less, 15% or less, or 10% or less, and preferably 40% or less.

The cell population to be used in producing the composition of the present disclosure may comprise cells other than skeletal myoblasts and fibroblasts but the content of the other cells is preferably as low as possible. Accordingly, the higher the total value of the CD56 positive rate and the TE7 positive rate, the more preferable. The total CD56 and TE7 value is, for example, 80% or more, 85% or more, 90% or more, 95% or more, 96% or more, 97% or more, 98% or more or 99% or more and preferably 90% or more.

In an embodiment, the composition of the present disclosure may be applied to a tissue, for example, in combination with another composition and/or graft for promoting healing. Examples of the composition and/or graft for promoting healing include, but are not limited to, a graft comprising a vascular pedicle such as an omentum majus piece, fibrin gel and Adspray®. In a preferred embodiment, the composition of the present disclosure is applied together with a graft comprising a vascular pedicle. A representative example of the graft comprising a vascular pedicle is an omentum majus piece.

The composition and/or graft for promoting healing may be a composition independent of the composition of the present disclosure, or may be integrated into the composition of the present disclosure.

In a case where the composition of the present disclosure is applied in combination with another composition and/or a graft for promoting heating independently of the composition of the present disclosure, the composition and/or graft may be applied before or after the application of the composition of the present disclosure. In a case where the composition and/or graft is applied before the application of the composition of the present disclosure, the composition and/or graft is applied so as to be positioned between an application site and the composition of the present disclosure. More specifically, the composition and/or graft is first applied to an application site, and thereafter, the composition of the present disclosure is applied over the composition and/or graft. In a case where the composition and/or graft is applied after the application of the composition of the present disclosure, the composition and/or graft is applied to an application site with the composition of the present disclosure interposed between them. More specifically, the composition of the present disclosure is first applied to an application site, and then, the composition and/or graft is applied on the composition of the present disclosure.

A mechanism for tissue regeneration is considered, for example, due to a paracrine effect, which is bought by sustained-release of the composition of the present disclosure of e.g., cytokines such as VEGF, HGF and collagen, which play a role in, e.g., angiogenesis, and protection and repair of cells at an affected site; and/or, due to an autocrine effect, which is brought by e.g., collagen production promoted by activation of progenitor cells or stem cells of the tissue around the application site.

In a case where the composition of the present disclosure takes the form of a sheet-shaped cell culture, the size of the sheet-shaped cell culture of the present disclosure is not particularly limited as long as it can cover a predetermined part of living tissue. If the sheet-shaped cell culture is, for example, circular, the diameter thereof is 10 to 55 mm, 15 to 50 mm, 20 to 45 mm, 25 to 40 mm, or 30 to 35 mm.

### Method for Treating Liver Dysfunction:

Another aspect of the present disclosure relates to a method for treating liver dysfunction, including a step of applying the composition of the present disclosure to a site exhibiting liver dysfunction (sometimes referred to as “the treatment method of the present disclosure”).

In the present disclosure, the term “site exhibiting liver dysfunction” refers to a liver site at which liver function is impaired or suppressed and/or a liver site that lost liver function. In an embodiment, the site exhibiting liver dysfunction is a site of the liver surface at which liver function is impaired or suppressed and/or a site of the liver surface losing liver function. In an embodiment, the site exhibiting liver dysfunction is a site of the liver surface exhibiting hepatitis, liver fibrosis, liver cirrhosis, liver cancer, or liver failure. In an embodiment, the site exhibiting liver dysfunction is a site of the liver surface exhibiting compensated cirrhosis or decompensated cirrhosis. In an embodiment, the site exhibiting liver dysfunction is a site of the liver surface exhibiting initial- or terminal-stage decompensated cirrhosis In an embodiment, the site exhibiting liver dysfunction is a site of the surface of residual liver exhibiting liver failure after liver cancer resection.

In an embodiment, in the treatment method of the present disclosure, the step of applying the composition of the present disclosure to a site exhibiting liver dysfunction is performed by an implantation device. In an embodiment, the implantation device refers to a medical device enabling the composition of the present disclosure to apply to the liver surface. In a case where the composition of the present disclosure is a sheet-shaped cell culture, the implantation device is, for example, a medical device having a planar structure capable of supporting and detaching the sheet-shaped cell culture. In view of operating it within the abdominal cavity, the implantation device preferably has a form that can be inserted and passed through a cylindrical body which has been inserted in a body cavity in endoscopic surgery. In an embodiment, the implantation device is a catheter.

In the present disclosure, the term “catheter” refers to a medical tube having flexibility. The composition of the present disclosure can be applied to a site exhibiting liver dysfunction by inserting a catheter to, e.g., an abdominal cavity or a blood vessel. The composition of the present disclosure can be added, administered, injected, delivered, applied, attached, engrafted, and/or implanted to a site exhibiting liver dysfunction by inserting a catheter to, e.g., an abdominal cavity and a blood vessel.

In at least one embodiment, in the treatment method of the present disclosure, the step of applying the composition of the present disclosure to a site exhibiting liver dysfunction is a step of adding, administering, injecting delivering, applying, attaching, engrafting, and/or implanting the composition of the present disclosure to a site exhibiting liver dysfunction.

### Use in Producing Medicine for Treating Liver Dysfunction:

Another aspect of the present disclosure relates to use of the composition of the present disclosure in producing a medicine (sometimes referred to as “use in producing the medicine of the disclosure”) for treating liver dysfunction or improving liver function.

### Use for Treating Liver Dysfunction:

Another aspect of the present disclosure relates to use of the composition of the present disclosure, (sometimes referred to as “use of the present disclosure”) for treating liver dysfunction or improving liver function.

### Method for Promoting Proliferation and Others of Liver Cells:

Another aspect of the present disclosure relates to a method for promoting proliferation of liver cells, regeneration of the liver tissue and/or angiogenesis, including a step of co-culturing the composition of the present disclosure with liver cells, liver tissue, and/or endothelial cells (sometimes referred to as the method of the present disclosure for promoting e.g., proliferation of liver cells). The method of the present disclosure for promoting e.g., proliferation of liver cells, regeneration of the liver tissue and/or angiogenesis through cytokine production.

In the present disclosure, the “endothelial cells” are cells constituting the endothelium in which capillary formation is likely induced by a factor derived from skeletal myoblasts. In an embodiment, the endothelial cells are vascular endothelial cells constituting artery, vein, and/or capillaries. In an embodiment, the endothelial cells are liver endothelial cells. In one embodiment, the endothelial cells are sinusoidal endothelial cells. In one embodiment, the endothelial cells are the sinusoidal endothelial cells of the liver.

In the present disclosure, the “co-culture” refers to culturing a plurality of cells and/or a plurality of types of cells in the same environment.

In an embodiment, the method of the present disclosure for promoting, e.g., proliferation of liver cells is performed in vitro.

In an embodiment, the method of the present disclosure for promoting, e.g., proliferation of liver cells is performed, in vivo.

The present disclosure will be more specifically described with reference to the following Examples, which show specific examples of the present disclosure and which are not intended to limit the present disclosure.

### Examples

### Example 1—Production of Growth Factors Derived from Human Skeletal Muscle Myoblasts

To a culture container (e.g., an IWAKI tissue-culture dish (surface treated for adherent cells) (3020-100), by AGC TECHNO GLASS Co., Ltd.), Dulbecco's modified eagle medium (DMEM) (Nacalai Tesque Inc.) is added. Subsequently, 1.0× 10⁶ of human skeletal muscle myoblasts (HSMM) (Normal HSMM, Lonza K.K.) are seeded. Culture is performed at 37° C., in the condition of 5% CO₂. The culture medium after 3 days from the start of culture is centrifuged at 2,000×g for 10 minutes, and the supernatant is collected (n=3). The supernatant is subjected to quantification based on ELISA using a commercially available ELISA kit (e.g., Human VEGF ELISA Kit (ab222510) and Human HGF ELISA Kit (ab100534), Abcam) for a vascular endothelial growth factor (VEGF) and a hepatocyte growth factor (HGF). Furthermore, HSMM is cultured in the same manner as above and the supernatant of 7 days after the start of culture is collected (n=3) and subjected to quantification based on ELISA for each of VEGF and HGF. For the color reaction in ELISA, the optical density (OD) at 450 nm is measured by a microplate reader (e.g., 680 XR, Bio-Rad Laboratories, Inc.).

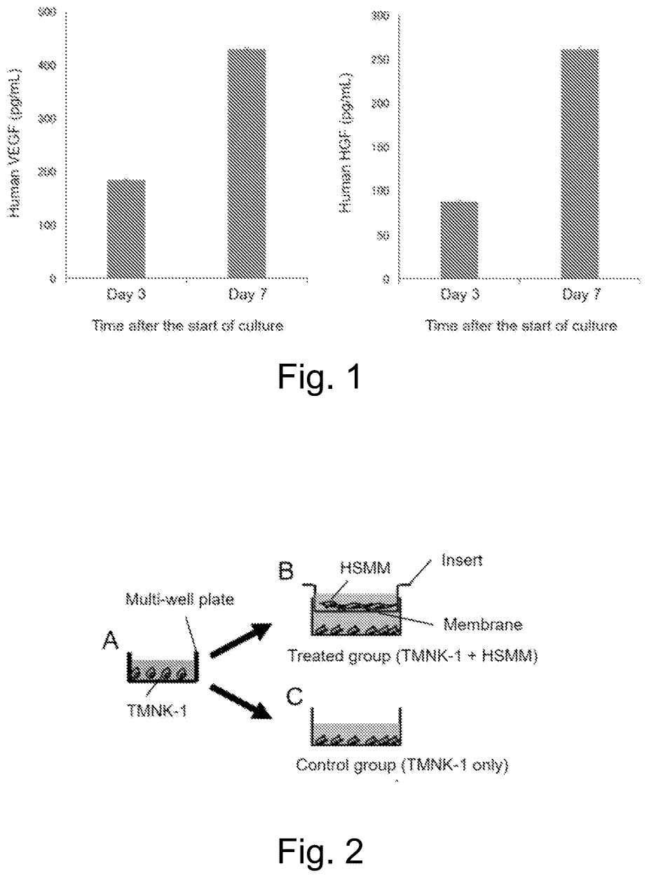

As a result, it is confirmed that the expression level of each of VEGF and HGF increases from 3 to 7 days after the start of culture (see, e.g., FIG. 1). VEGF is known to produce HGF from sinusoidal endothelial cells through the VEGFR2-ID1 signal transduction system in the sinusoidal endothelial cells of the liver. Furthermore, HGF is known to promote the proliferation of liver cells and the reproduction of liver tissue. Thus, it is found that HSMM promotes the proliferation of liver cells and the reproduction of liver tissue through cytokine production such as VEGF and HGF.

### Example 2—Induction of Capillary Formation by Factor Derived from Human Skeletal Muscle Myoblasts

### (1) Preparation of Immortalized Human Liver Endothelial Cells (TMNK-1)-Comprising Medium:

To a Transwell® multiwell plate (6.5 mm, attached with 0.4 um Pore Polyester Membrane Insert, Corning), Dulbecco's modified eagle medium (DMEM) (Nacalai Tesque Inc.) is added, and subsequently, 1.0× 10⁵ of immortalized human liver endothelial cells (TMNK-1) (provided by, for example, JCRB cell bank, cell number (JCRB): JCRB1564) are seeded (TMNK-1-comprising medium) (see, e.g., “A” of FIG. 2).

### (2) Preparation of Human Skeletal Myoblast (HSMM)-Comprising Medium:

To a Transwell® insert, DMEM is added and subsequently, 1.0×10⁵ of human skeletal myoblasts (HSMM) (Normal HSMM, Lonza K.K.) are seeded (HSMM (1.0×10⁵ cells)-comprising medium). Separately, to a Transwell® insert, DMEM is added and subsequently, 5.0×10⁵ of HSMM are seeded (HSMM (5.0×10⁵ cells)-comprising medium).

### (3) Soaking HSMM-Comprising Medium in TMNK-1-Comprising Medium:

The Transwell® insert, which comprises HSMM (1.0×10⁵ cells)-comprising medium and is prepared according to (2) above, is inserted in the Transwell® multiwell plate, which comprises TMNK-1-comprising medium and is prepared according to (1) above. In this manner, the HSMM-comprising medium is soaked in the TMNK-1-comprising medium and designated as treated group 1 (n=3) (see, e.g., “B” of FIG. 2). Similarly, the Transwell® insert, which comprises HSMM (5.0×10⁵ cells)-comprising medium and is prepared according to (2) above, is inserted in the Transwell® multiwell plate, which comprises TMNK-1-comprising medium and is prepared according to (1) above. In this manner, the HSMM-comprising medium is soaked in the TMNK-1-comprising medium and designated as treated group 2 (n=3) (see, e.g., “B” of FIG. 2). The soaking makes it possible to exchange various factors between the HSMM-comprising medium and TMNK-1-comprising medium through the membrane of the Transwell® insert. A Transwell® multiwell plate, which comprises the TMNK-1-comprising medium, is prepared according to (1) above and not soaked, is used as a control group (n=3) (see, e.g., “C” of FIG. 2).

### Induction of Capillary Formation:

Treated group 1, treated group 2, and the control group described in the above (3) are cultured at 37° C. in the condition of 5% CO₂. One hour, 5 hours and 24 hours after the start of culture, the Transwell® insert of each of treated group 1 and treated group 2 is removed. TMNK-1 contained in each of Transwell® multiwell plates of the treated group 1, treated group 2, and the control group is observed by an optical microscope at a 200-fold magnification.

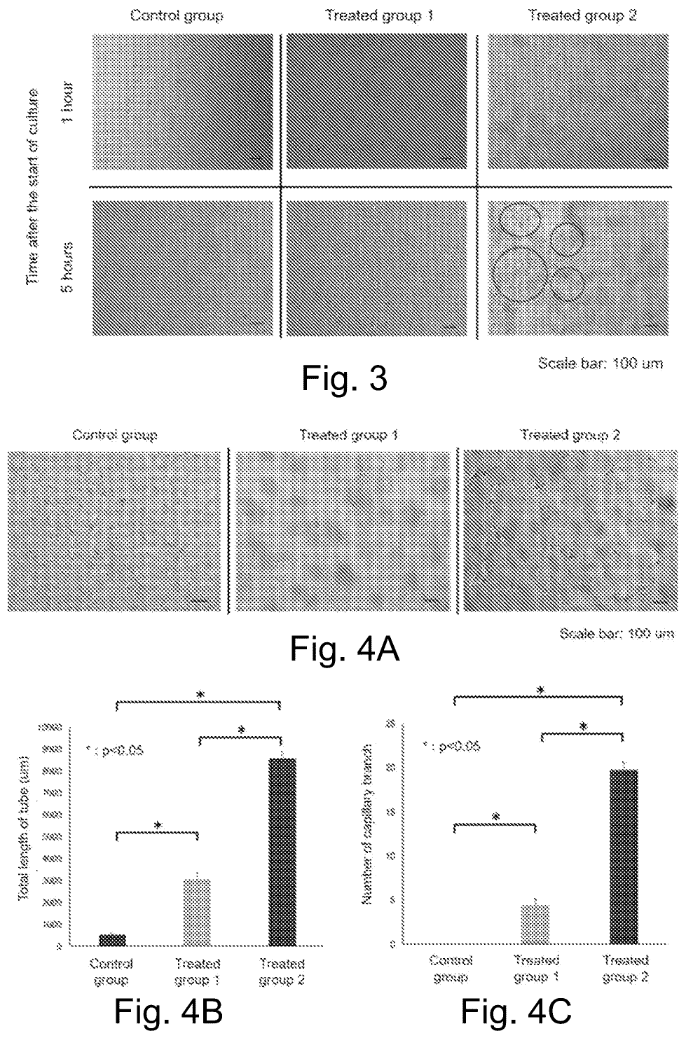

As a result, it is confirmed that TMNK-1 capillary formation is induced by HSMM, 5 hours after the start of culture in treated group 2 compared to the control group (see, e.g., FIG. 3). Furthermore, it is confirmed that TMNK-1 capillary formation is induced by HSMM, 24 hours after the start of culture in treated groups 1 and 2 compared to the control group (see, e.g., FIG. 4A). Moreover, it is confirmed that the total length of the capillary in treated group 2 is longer and the number of branches thereof are larger than treated group 1, 24 hours after the start of culture (see, e.g., FIGS. 4B and 4C). More specifically, it is confirmed that the larger the number of HSMM cells in the HSMM-comprising medium, the more actively the capillary formation of TMNK-1 is induced by HSMM. Thus, it is found that TMNK-1 capillary formation is induced by the factor derived from HSMM; in other words, angiogenesis is promoted.

From the results in the foregoing, according to the present disclosure, liver dysfunction such as hepatitis, liver fibrosis, liver cirrhosis, liver cancer, or liver failure can be treated. In particular, according to the present disclosure, it is possible to treat terminal-stage decompensated cirrhosis whose improvement cannot be expected by current internal therapy as well as to treat liver failure of residual liver after liver cancer resection. Furthermore, according to the present disclosure, it is possible to promote proliferation of the liver cells, regeneration of the liver tissue and/or angiogenesis. Moreover, according to the present disclosure, it is possible to treat liver dysfunction through cytokine production.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

It is to be appreciated that any feature described herein can be claimed in combination with any other feature(s) as described herein, regardless of whether the features come from the same described embodiment.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A composition comprising skeletal myoblasts for treating liver dysfunction or improving liver function.

2. The composition of claim 1, wherein the liver dysfunction is hepatitis, liver fibrosis, liver cirrhosis, liver cancer, or liver failure.

3. The composition of claim 1, wherein the liver dysfunction is liver cirrhosis.

4. The composition of claim 1, wherein the liver dysfunction is liver cancer.

5. The composition of claim 4, for application to a liver after resection of liver cancer.

6. The composition of claim 1, for promoting proliferation of liver cells, regeneration of a liver tissue and/or angiogenesis.

7. The composition of claim 1, for treating liver dysfunction through cytokine production.

8. A method for treating liver dysfunction, comprising, a step of applying the composition of claim 1 to a site exhibiting liver dysfunction.

9. The method according to claim 8, wherein the step of applying the composition to a site exhibiting liver dysfunction is performed by an implantation device.

10. The method according to claim 9, wherein the implantation device is a catheter.

11. A method for treating for treating organ dysfunction, comprising:
identifying a site of dysfunction in an organ;
producing a composition comprising skeletal myoblasts based at least in part on the identifying; and
applying the composition to the site exhibiting the organ dysfunction via one or more of adding, administrating, injecting, delivering, applying, attaching, engrafting, and implanting.

12. The method of claim 11, wherein the composition further comprises adhesive somatic cells, stem cells other than skeletal myoblasts, or both in combination with the skeletal myoblasts, and wherein the composition comprises at least about 50% of the skeletal myoblasts.

13. The method of claim 11, wherein the composition is in a liquid-state and is substantially flowable for application to the site.

14. The method of claim 11, wherein the composition is in a solid-state and is substantially flexible for application to the site.

15. The method of claim 11, comprises:
co-culturing the composition, in vitro, with cells or tissue associated with the organ exhibiting dysfunction, endothelial cells, or a combination thereof.

16. The method of claim 11, comprises:
co-culturing the composition, in vivo, with cells or tissue associated with the organ exhibiting dysfunction, endothelial cells, or a combination thereof.

17. The method of claim 11, comprises:
promoting cytokine production, proliferation of cells, regeneration of tissue, angiogenesis, or a combination thereof associated with the organ exhibiting dysfunction.

18. A method for producing a cell-comprising composition for use in treating organ dysfunction, comprising:
seeding a cell population comprising skeletal myoblasts on a culture substrate.

19. The method of claim 18, wherein the cell-comprising composition further comprises adhesive somatic cells, stem cells other than skeletal myoblasts, or both in combination with the skeletal myoblasts, and wherein the cell-comprising composition comprises at least about 50% of the skeletal myoblasts.

20. The method of claim 18, wherein the cell-comprising composition is in a liquid-state and is substantially flowable for application to a site of organ dysfunction; or
wherein the cell-comprising composition is in a solid-state and is substantially flexible for application to the site of organ dysfunction.
